(19) United States
(12) Patent Application Publication
(10) Pub. No.: US 2009/0054728 A1
(43) Pub. Date: Feb. 26, 2009

(54) Manipulatable guide system and methods for natural orifice translumenal endoscopic surgery

(76) Inventor: Robert M. Trusty, Cincinnati, OH (US)

Correspondence Address: K&L GATES LLP, 535 SMITHFIELD STREET, PITTSBURGH, PA, 15222, US

(21) Appl. No.: 11/894,358

(22) Filed: Aug. 21, 2007

(51) Int. Cl. A61B 1/01

(52) U.S. Cl. 600/114

(57) ABSTRACT

A guide system for accommodating, supporting and manipulating endoscopic tools. In various embodiments, the guide system includes a hollow outer sheath that may have a steerable distal end. The system may further include inner sheaths that are configured relative to the outer sheath to enable the inner sheaths to be inserted one at a time into the outer sheath and moved therein. The inner sheaths may be configured with different numbers, sizes and shapes of working channels for accommodating a variety of different endoscopic tools. The device may also be equipped with seals such that the inner sheath as well as the various endoscopic tools supported by the inner sheath may be manipulated within the body cavity while maintaining insufflation thereof.

### FIELD OF THE INVENTION

The present invention relates, in general, to guide tubes for endoscopes and medical procedures and, more particularly, to devices for facilitating the insertion and manipulation of endoscopes and other surgical implements within a body cavity to accomplish various surgical and therapeutic procedures.

### BACKGROUND OF THE INVENTION

Minimally invasive procedures are desirable because such procedures can reduce pain and provide relatively quick recovery times as compared with conventional open medical procedures. Many minimally invasive procedures are performed through one or more ports through the abdominal wall, commonly known as trocars. A laparascope that may or may not include a camera may be used through one of these ports for visualization of the anatomy and surgical instruments may be used simultaneously through other ports. Such devices and procedures permit a physician to position, manipulate, and view anatomy, surgical instruments and accessories inside the patient through a small access opening in the patient's body.

Still less invasive procedures include those that are performed through insertion of an endoscope through a natural body orifice to a treatment region. Examples of this approach include, but are not limited to, cystoscopy, hysteroscopy, esophagogastroduodenoscopy, and colonoscopy. Many of these procedures employ the use of a flexible endoscope during the procedure. Flexible endoscopes often have a flexible, steerable articulating section near the distal end that can be controlled by the user by utilizing controls at the proximal end. Treatment or diagnosis may be completed intralumenally, such as polypectomy or gastroscopy. Alternatively, treatment or diagnosis of extra-luminal anatomy in the abdominal cavity may be completed translumenally, for example, through a gastrotomy, colonotomy or vaginotomy. Minimally invasive therapeutic procedures to treat or diagnose diseased tissue by introducing medical instruments translumenally to a tissue treatment region through a natural opening of the patient are known as Natural Orifice Translumenal Endoscopic Surgery (NOTES)™.

Some flexible endoscopes are relatively small (1 mm to 3 mm in diameter), and may have no integral accessory channel (also called biopsy channels or working channels). Other flexible endoscopes, including gastroscopes and colonoscopes, have integral working channels having a diameter of about 2.0 to 3.5 mm for the purpose of introducing and removing medical devices and other accessory devices to perform diagnosis or therapy within the patient. As a result, the accessory devices used by a physician can be limited in size by the diameter of the accessory channel of the scope used. Additionally, the physician may be limited to a single accessory device when using the standard endoscope having one working channel.

Over the years, a variety of different sheaths and overtubes for accommodating endoscopes and the like have been developed. Some sheath arrangements such as those disclosed in U.S. Pat. No. 5,325,845 to Adair are substantially steerable by means of control knobs supported on a housing assembly. Regardless of the type of surgery involved and the method in which the endoscope is inserted into the body, the surgeons and surgical specialists performing such procedures have generally developed skill sets and approaches that rely on anatomical alignment for both visualization and tissue manipulation purposes. However, due to various limitations of those prior overtube and sheath arrangements, the surgeon may often times be forced to view the surgical site in such a way that is unnatural and thereby difficult to follow and translate directional movement within the operating theater to corresponding directional movement at the surgical site. Moreover, such prior devices are not particularly well-equipped to accommodate and manipulate multiple surgical instruments and tools within the surgical site without having to actually move and reorient the overtube.

Consequently a significant need exists for an alternative to conventional overtubes and sheaths for use with endoscopes and other surgical tools and instruments that can be advantageously manipulated and oriented and which can accommodate a variety of different tools and instruments and facilitate movement and reorientation of such tools and instruments without having to reorient or move the outer sheath.

The foregoing discussion is intended only to illustrate some of the shortcomings present in the field of the invention at the time, and should not be taken as a disavowal of claim scope.

### SUMMARY

In one general aspect of the invention, there is provided guide system for accommodating endoscopic tools. In various embodiments, the guide system may comprise a hollow outer sheath that has a proximal end and a distal end. The distal end may be substantially steerable. The system may further include an inner sheath that has a proximal end and a distal end and is sized relative to the outer sheath to permit the inner sheath to be selectively rotated and axially moved within the hollow outer sheath such that the distal end of the inner sheath may selectively protrude beyond the distal end of the outer sheath and wherein the inner sheath has at least one working channel formed therein.

In another general aspect of various embodiments of the present invention there is provided a guide system for accommodating endoscopic tools. In various embodiments, the guide system may comprise a hollow outer sheath that has a proximal end and a distal end. The distal end may be substantially steerable. The system may further include a plurality of inner sheaths that each may be selectively inserted into the outer sheath one at a time. Each inner sheath may have a proximal end and a distal end and be sized relative to the outer sheath to permit the inner sheath to be selectively rotated and axially moved within the outer sheath such that the distal end of the inner sheath may selectively protrude beyond the distal end of the outer sheath and wherein the inner sheaths are differently configured from each other to operably support different tool configurations therein.

These and other objects and advantages of the present invention shall be made apparent from the accompanying drawings and the description thereof.

### BRIEF DESCRIPTION OF THE FIGURES

The accompanying drawings, which are incorporated in and constitute a part of this specification, illustrate embodiments of the invention, and, together with the general description of the invention given above, and the detailed description of the embodiments given below, serve to explain various principles of the present invention.

FIG. 1 is a side view of a guide system embodiment of the present invention;

FIG. 2 is a side view of an inner sheath embodiment of the present invention;

FIG. 3 is a partial perspective view of a distal end portion of an inner sheath embodiment of the present invention;

FIG. 4 is a partial perspective view of a distal end portion of an outer sheath of the present invention;

FIG. 5 is a partial perspective view of the inner sheath embodiment of FIG. 3 inserted in the outer sheath of FIG. 4;

FIG. 6 is a partial perspective view of a distal end portion of another inner sheath embodiment of the present invention;

FIG. 7 is an end view of another inner sheath embodiment of the present invention inserted into an outer sheath embodiment of the present invention;

FIG. 8 is a partial perspective view of a distal end portion of another inner sheath embodiment of the present invention that has locking detents formed thereon;

FIG. 9 is a partial perspective view of a distal end portion of another outer sheath of the present invention with detent pockets formed therein;

FIG. 10 is a partial perspective view of the inner sheath embodiment of FIG. 8 inserted in the outer sheath embodiment of FIG. 9;

FIG. 11 is a diagrammatical view illustrating the use of one embodiment of a guide system of the present invention inserted through a patient's mouth and esophagus to perform a gastrotomy through the stomach wall;

FIG. 12 is another diagrammatical view of the guide system and patient's stomach of FIG. 11, with a conventional hole-forming device extending through a conventional endoscope supported in the guide system and forming a hole through the stomach wall;

FIG. 13 is another diagrammatical view of the guide system and patient's stomach depicted in FIGS. 11 and 12, with the inner sheath of the guide system protruding out of the outer sheath; and

FIG. 14 is another diagrammatical view of the guide system and patient's stomach after a portion of the body cavity has been insufflated.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the various embodiments of the present invention is defined solely by the claims. The features illustrated or described in connection with one exemplary embodiment may be combined with the features of other embodiments. Such modifications and variations are intended to be included within the scope of the present invention.

The present invention generally relates to various devices and overtube arrangements for use in connection with surgical instruments such as, for example, endoscopes for selectively positioning and manipulating endoscopic tools in a desired orientation within the body cavity. The term “endoscopic tools”, as used herein may comprise, for example, endoscopes, lights, insufflation devices, cleaning devices, suction devices, hole-forming devices, imaging devices, cameras, graspers, clip appliers, loops, Radio Frequency (RF) ablation devices, harmonic ablation devices, scissors, knives, suturing devices, etc. However, such term is not limited to those specific devices. As the present Detailed Description proceeds, those of ordinary skill in the art will appreciate that the unique and novel features of the various instruments and methods of the present invention may be effectively employed to perform surgical procedures by inserting such endoscopic tools through a natural body lumen (mouth, anus, vagina, etc.) to perform surgical procedures within a body cavity.

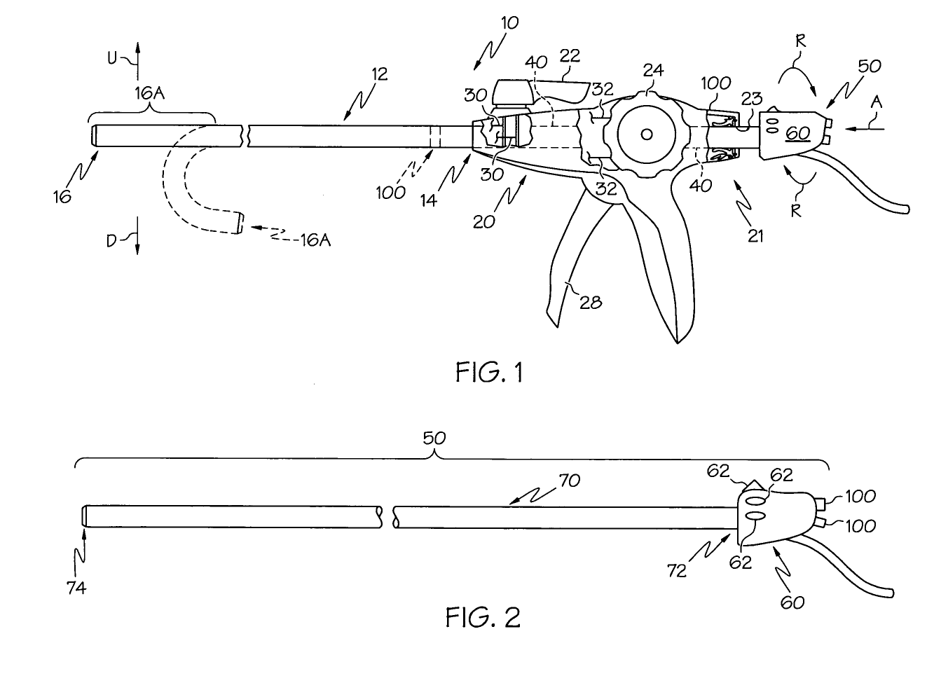

FIG. 1 illustrates an embodiment of a guide system 10 of the present invention that comprises an outer sheath 12 that has a proximal end 14 coupled to a handle assembly 20. It will be appreciated that the terms “proximal” and “distal” are used herein with reference to a clinician manipulating the handle assembly 20. The term “proximal” referring to the portion closest to the clinician and the term “distal” referring to the portion located away from the clinician. It will be further appreciated that for convenience and clarity, spatial terms such as “vertical”, “horizontal”, “up” and “down” may be used herein with respect to the drawings. However, surgical instruments are used in many orientations and positions, and these terms are not intended to be limiting and absolute.

As can be seen in FIG. 1, the elongated hollow outer sheath 12 may further have a distal end 16 that is substantially steerable by control knobs 22 and 24 operably supported on the handle assembly 20. For example, the control knob 22 may be operably coupled to a first pair of right/left cables 30 that extend through lumens (not shown) in the outer sheath 14 and are operably affixed to the distal end 16 of the outer sheath 14. Similarly, the control knob 24 may be operably affixed to up/down cables 32 that also extend through corresponding lumens (not shown) in the outer sheath 14 and are affixed to the distal end 16 thereof. Thus, rotation of the control knob 22 relative to the handle assembly 20 may cause the distal end 16 of outer sheath 12 to move in left and right directions (into and out of the page as depicted in FIG. 1) and rotation of the control knob 24 relative to the handle assembly 20 may cause the distal end 16 of the hollow outer sheath 12 to move up and down (arrows “U” and “D” in FIG. 1). A locking trigger 28 may be provided to selectively lock the distal end 16 in a desired position. Steerable sheaths and tube arrangements are known in the art and, therefore, their construction and use will not be discussed in great detail herein. For example, U.S. patent application Ser. No. 11/762,855 to James T. Spivey and Omar J. Vakharia, entitled CONTROL MECHANISM FOR FLEXIBLE ENDOSCOPE DEVICE AND METHOD OF USE, filed Jun. 14, 2007 which is commonly owned by the Assignee of the present application discloses such an arrangement and is herein incorporated by reference in its entirety. Another steerable sheath arrangement is disclosed in U.S. Pat. No. 5,325,845 to Aidar, issued Jul. 5, 1994, the entire disclosure of which is herein incorporated by reference.

In various embodiments, the hollow outer sheath 12 may be fabricated from, for example, plastic, Teflon® or rubber inner/outer sheath material and a metallic, plastic, or composite coil pipe or extruded insertion tube which may provide some axial and rotational stiffness to allow for push/pull and rotation of the outer sheath. The articulation section 16A may be fabricated from, for example, a series of joined plastic, metallic, or composite links or from a plastic, metallic or composite tube with material removed in locations to allow articulation of the distal end 16 thereof in two axes and surrounded with material similar to the remainder of the outer sheath 12. The proximal end 14 of the hollow outer sheath 12 may be substantially coaxially aligned with a lumen 40 that extends through the handle assembly 20 such that an inner sheath assembly 50 may be inserted through an opening 23 in the proximal end 21 of the handle assembly 20, through lumen 40 and into the hollow outer sheath 12 as illustrated in FIG. 1. In various embodiments, the inner sheath assembly 50 comprises a control head 60 that has a substantially flexible inner sheath 70 attached thereto. The inner sheath may be fabricated from, for example, plastic, Teflon® or rubber inner/outer sheath material and a metallic, plastic, or composite coil pipe or extruded insertion tube and have a proximal end 72 that is attached to the control head 60. The inner sheath 70 may further have a distal end 74 and be configured relative to the hollow outer sheath 12 such that the inner sheath 70 may be selectively rotatable and axially movable within the outer sheath 12 as represented by arrows “A” and “R” in FIGS. 1 and 5. The inner sheath 70 may also be sized and configured relative to the outer sheath 12, for example, such that the distal end 74 of the inner sheath 70 may protrude out beyond the distal end 16 of the outer sheath 12 as shown in FIG. 5. Those of ordinary skill in the art will appreciate that such arrangement not only enables the distal end 74 of the inner sheath 70 to be advantageously positioned, but the distal end 74 can also be used to move and manipulate tissue as needed.

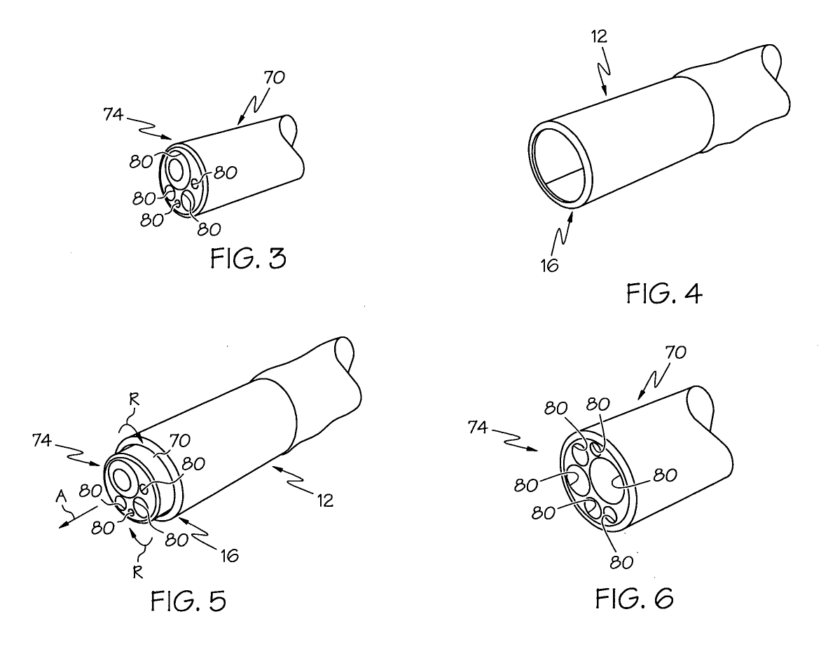

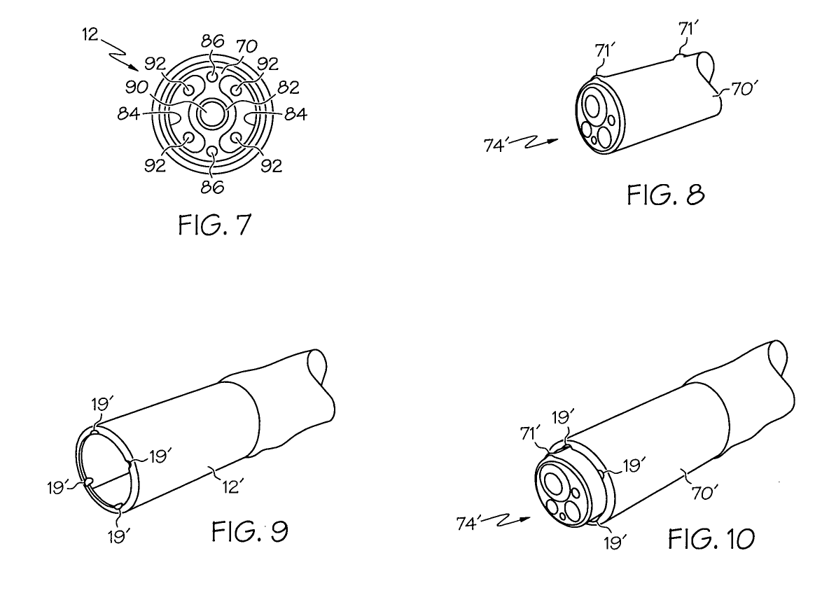

As can be seen in FIGS. 3 and 5, the inner sheath 70 may have at least one, and preferably a plurality of, working channels 80 formed therein. The working channels 80 may vary in number, size, and shape. For example, in the embodiment depicted in FIG. 3, the inner sheath 70 has five working channels 80 therein that vary in size, but all have a substantially circular cross-section. In the embodiment depicted in FIG. 6, the inner sheath 70 has six working channels 80 of various sizes. In the embodiment depicted in FIG. 7, the inner sheath 70 has a somewhat “honeycombed” cross-sectional configuration. In that embodiment, a central lumen or working channel 82 is provided though the inner sheath 70. Such central lumen 82 may, for example, operably support a camera 90 therein. Oriented around the central lumen 82 are two “oblong” working channels 84 that may, for example, each support a plurality of endoscopic tools 92 (hole-forming devices, light bundles, imaging devices, cameras, graspers, clip appliers, loops, Radio Frequency (RF) ablation devices, harmonic ablation devices, scissors, knives, suturing devices, etc.). This embodiment also includes smaller working channels 86 that may facilitate the introduction of an insufflation medium (for example, air or carbon dioxide, fluid, such as, for example, water, saline solution, sterile solution, alcohol, betadine, staining inks, staining dyes into the body area adjacent the target tissue.

In some applications, it may be advantageous to essentially lock the inner sheath in a predetermined position relative to the outer sheath. For example, as can be seen in FIGS. 8-10, the inner sheath 70′ may have one or more than one detents 71′ formed thereon that may be received in corresponding pockets 19′ provided in the distal end 16′ of the outer sheath 12′. Thus, the inner sheath 70′ may be rotated to a predetermined position defined by the corresponding pockets 19′ and retained in that position relative to outer sheath 12′ by bringing the corresponding detent 71′ into locking engagement with the corresponding pocket 19′. Those of ordinary skill in the art will understand that such locking arrangement may be provided in a variety of different forms without departing from the spirit and scope of the present invention. For example, in an alternative embodiment, the detents may be provided in the outer sheath and the pockets may be provided in the inner sheath. In other embodiments, the detents may extend substantially the entire length of the sheath and the pockets may each comprise an axial groove that also extends substantially the entire length of the sheaths. Different numbers, shapes and sizes of detents and/or pockets may also be employed.

In various embodiments, one or more seals 100 may be employed to achieve a substantially airtight/fluidtight seal around the inner sheath 70. For example, a seal 100 may be provided in the handle assembly 100 to achieve an airtight/fluidtight seal between the inner sheath 70 and the lumen 40 in the handle assembly 20. In addition to, or in the alternative, a seal 100 may be provided in the outer sheath 12 to achieve a substantially fluidtight or airtight seal between the inner sheath 70 and the outer sheath 12. A variety of existing seal arrangements may be employed. For example, U.S. Pat. No. 5,401,248, entitled SEAL FOR TROCAR ASSEMBLY, issued Mar. 28, 1995 to Bencini and U.S. Pat. No. 7,163,525, entitled DUCKBILL SEAL PROTECTOR, issued Jan. 16, 2007, the disclosures of which are each herein incorporated by reference in their respective entireties disclose seals that may be employed to establish a substantially airtight/fluidtight seal between the inner sheath 70 and outer sheath 12. The working channels 80 in the inner sheath 70 may also each be fitted with a similar seal 100 such that when the working channel 80 is not being used, the working channel 80 is sealed off and when an endoscopic tool is inserted into the working channel 80, a substantially airtight/fluidtight seal is achieved between the endoscopic tool and the working channel 80. In various embodiments, for example, the seals 100 may be mounted on the control head 60 as shown in FIG. 2.

The working channels 80, 84, 86 may be used to apply suction, pressurized air, fluid, etc. to an area within the body. The control head 60 of the inner sheath assembly 50 may be provided with a series of control buttons 62 or the like that serve to control various endoscopic tools or instruments inserted therethrough. For example, such control buttons 62 may be used to control the application of suction, insufflation mediums, cleaning mediums, etc. Such buttons may also consist of buttons for controlling lights, zooming of the camera, etc.

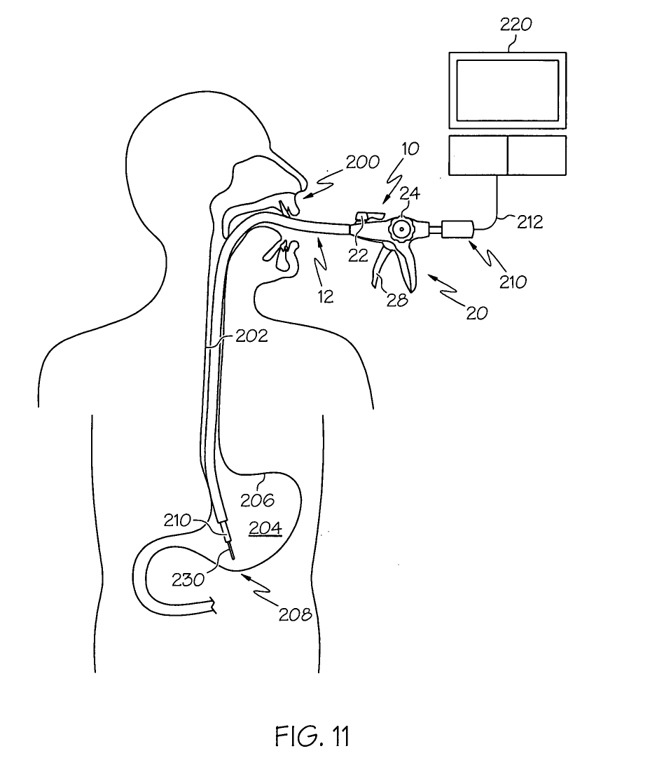

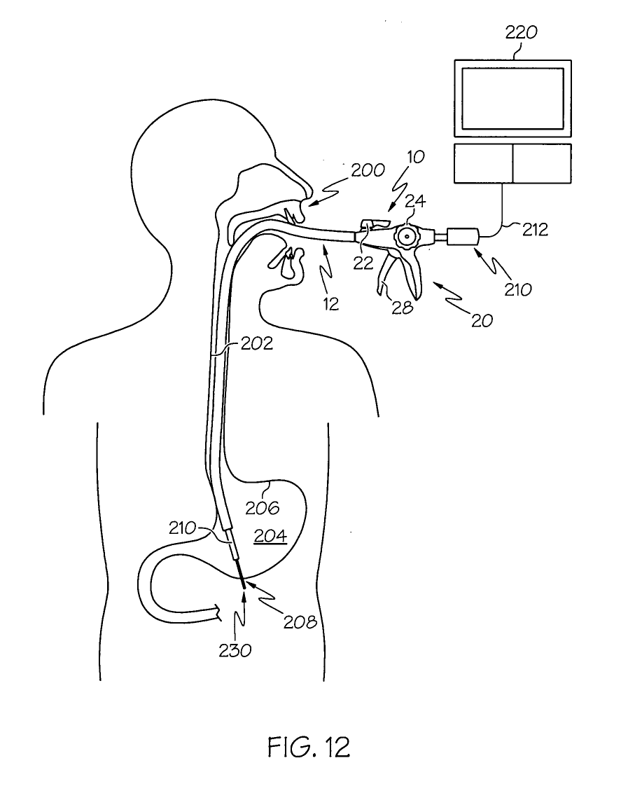

FIGS. 11-14 illustrate various methods of using the guide system 10 of the present invention. As can be seen in FIG. 11, the outer sheath 12 can be inserted through a natural orifice to form an opening through the stomach wall 206. In the example depicted in FIGS. 11-14, the outer sheath 12 is inserted through the mouth 200 and esophagus 202 into the stomach 204 to form an opening through the stomach wall 206. During this procedure, the clinician may manipulate the distal end 16 of the outer sheath 12 by means of the control knobs 22 and 24 as needed. Once the outer sheath 12 has been oriented in a desired position, the clinician may lock the outer sheath 12 in that position by engaging the locking trigger 28 on the handle assembly 20. The clinician may insert a conventional active or passive endoscope 210 that has a camera and a working channel therein through the outer sheath 12 as shown in FIG. 11 to locate the portion of the stomach wall 206 (or target tissue 208) through which the hole is to be made. The endoscope 210 may be attached to a viewing screen 220 in the operating suite by an umbilical cord 212. Once the target tissue 208 has been located and the endoscope 210 properly positioned, the clinician may insert a conventional hole-forming instrument 230 through the working channel in the endoscope 210 to form a hole 209 through the target tissue 208. See FIG. 12. After the hole 209 has been formed through the target tissue 208 and the outer sheath has been inserted through the hole, the endoscope 210 and hole-forming instrument 230 may be removed from the outer sheath 12.

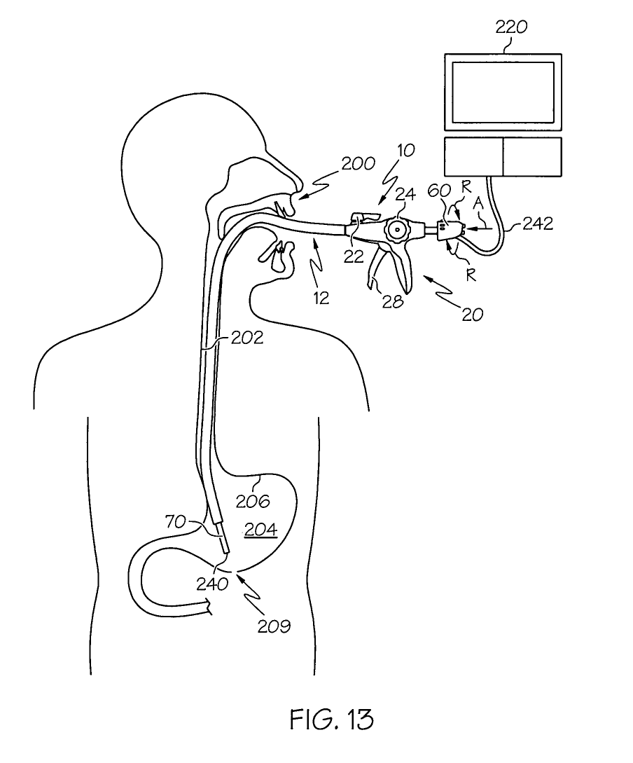

The clinician may then insert the inner sheath 70 in through the outer sheath 12 as shown in FIG. 13. A smaller camera 240 may be supported in one of the working channels in the inner sheath 70 and be coupled to the screen 220 by an umbilical cord 242. The distal end 74 of the inner sheath 70 may be axially advanced out of the distal end 16 of the outer sheath 12 as shown in FIG. 13 and rotated as necessary until the clinician attains a desired or familiar picture orientation on the screen 220. During this process, the clinician may use the distal end 74 of the inner sheath 70 to manipulate/position tissue as needed. Once in a desired position, the clinician may lock the inner sheath 70 relative to the outer sheath 12 by bringing the detent(s) into retaining engagement with corresponding pocket(s). Those of ordinary skill in the art will appreciate that the smaller camera 240 may also be advanced out through the distal end 74 of the inner sheath 70 as necessary.

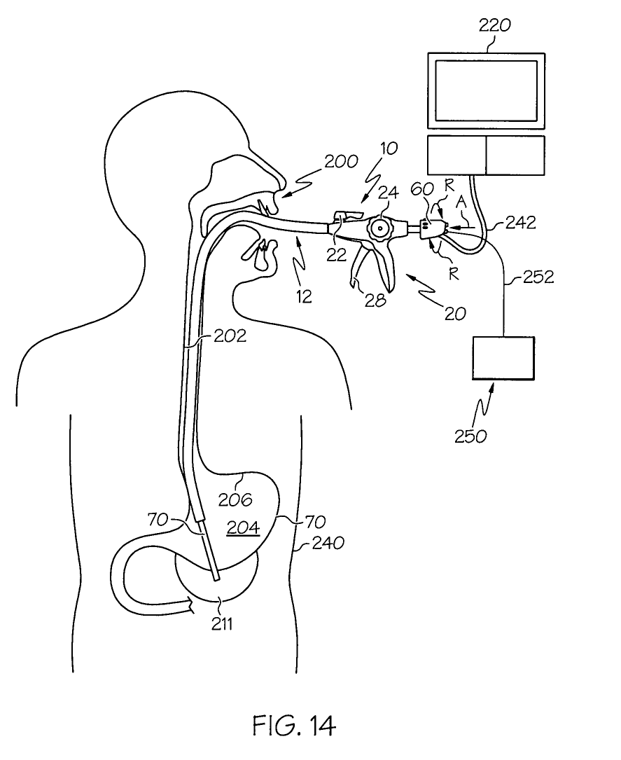

The medical procedure may further require the portion of the body cavity 211 adjacent to the target tissue 208 to be insufflated. To accomplish this procedure, an insufflation medium such as, for example, air or carbon dioxide may be introduced into the body cavity portion 211 through a working channel in the inner sheath 70. Such insufflation medium may be supplied through a supply line 252 that has been inserted into a working channel in the inner sheath 70 and is coupled to a source of insufflation medium 250. The insufflation medium is supplied through the supply line 252 extending through the working channel and, once the desired pressure is attained, a standard operating room insufflation controller can be used to maintain the desired pressure via the supply line 252. See FIG. 14. The clinician may then insert other endoscopic tools through the working channels in the inner sheath 70 to perform various procedures. The person of ordinary skill in the art will understand that the various seal arrangements employed in the guide system 10 facilitate maintenance of the insufflation within cavity portion 211 while additional tool(s)/instrument(s) are inserted and manipulated therein. It will be further appreciated that the inner sheath 70 may also be advantageously repositioned, axially moved, rotated, etc. during the operation as need to provide the clinician with the desired tool/instrument positioning and support as well as the desired video display orientation on the screen 220. This feature may be particularly useful to the clinician who is most familiar with a particular tissue orientation, for example, the tissue orientation that is often depicted in medical journals, books and reference materials or commonly addressed through open or laparoscopic surgical means.

While the present invention has been illustrated by description of several embodiments and while the illustrative embodiments have been described in considerable detail, it is not the intention of the applicant to restrict or in any way limit the scope of the appended claims to such detail. Additional advantages and modifications may readily appear to those skilled in the art. The guide system embodiments of the present invention represent vast improvements over prior overtube and sheath arrangements. Not only can the system allow the clinician to attain a desired viewing orientation during the operation and while maintaining desired insufflation of the area, the guide system also provides the added flexibility for accommodating instrument exchanges, instruments of various sizes and, if necessary, extraction of relatively large portions of tissue therethrough. In addition, the ability to freely move the inner sheath relative to the outer sheath (when unlocked) and also the ability to freely move the endoscopic tools within the inner and outer sheaths provide the clinician with the ability to use such instruments to manipulate and treat tissue as needed.

Furthermore, a variety of different inner sheath configurations may be employed with a single outer sheath/handle assembly arrangement to enable the clinician to perform a variety of different surgical procedures. For example, an inner sheath may have a specific number of appropriately sized working channels that are specifically suited for a particular procedure. The guide system may include several of such inner sheaths, such that the system may be advantageously used to perform several different surgical procedures, simply by using the appropriately configured inner sheath(s).

Those of ordinary skill in the art will also understand that the guide system may effectively employ a variety of different camera arrangements. For example, to further enhance the surgical experience, a camera may be employed that has zoom capability (either digital or optical). Such camera may be employed to mimic laparoscopic capabilities associated with moving a laparoscope during laparoscopic surgery for example, to provide a stadium view and a detailed view of the tissue as required by the clinician.

While several embodiments of the invention have been described, it should be apparent, however, that various modifications, alterations and adaptations to those embodiments may occur to persons skilled in the art with the attainment of some or all of the advantages of the invention. For example, according to various embodiments, a single component may be replaced by multiple components, and multiple components may be replaced by a single component, to perform a given function or functions. This application is therefore intended to cover all such modifications, alterations and adaptations without departing from the scope and spirit of the disclosed invention as defined by the appended claims.

The devices disclosed herein can be designed to be disposed of after a single use, or they can be designed to be used multiple times. In either case, however, the device can be reconditioned for reuse after at least one use. Reconditioning can include a combination of the steps of disassembly of the device, followed by cleaning or replacement of particular pieces, and subsequent reassembly. In particular, the device can be disassembled, and any number of particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those of ordinary skill in the art will appreciate that the reconditioning of a device can utilize a variety of different techniques for disassembly, cleaning/replacement, and reassembly. Use of such techniques, and the resulting reconditioned device, are all within the scope of the present application.

Preferably, the invention described herein will be processed before surgery. First a new or used instrument is obtained and, if necessary, cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or higher energy electrons. The radiation kills bacteria on the instrument and in the container. The sterilized instrument can then be stored in the sterile container. The sealed container keeps the instrument sterile until it is opened in the medical facility.

Those of ordinary skill in the art will appreciate that the devices disclosed herein may be provided in a kit that may, for example, be directed to a particular surgical procedure. For example, a kit may include a guide system 10 of the present invention in combination with a disposable endoscope that may or may not have a working channel therein. The guide system 10 may include a steerable outer sheath 12 and handle assembly 20 as well as at least one inner sheath 70 with a working channel configuration that may be particularly well-suited to accommodate those endoscopic tools likely to be employed during a particular surgical procedure. In other embodiments, the kit may include a plurality of inner sheaths 70 that each have different working channel configurations therein. Such kit arrangements provide the clinician with the added flexibility to select the appropriate inner sheath 70 for a particular procedure and to remove and insert other inner sheaths 70 with different working channels that are better suited to accommodate different endoscopic tools as the surgical procedure progresses.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

The invention which is intended to be protected is not to be construed as limited to the particular embodiments disclosed. The embodiments are therefore to be regarded as illustrative rather than restrictive. Variations and changes may be made by others without departing from the spirit of the present invention. Accordingly, it is expressly intended that all such equivalents, variations and changes which fall within the spirit and scope of the present invention as defined in the claims be embraced thereby.

What is claimed is:

1. A guide system for accommodating endoscopic tools, comprising:
a hollow outer sheath having a proximal end and a distal end, said distal end being substantially steerable; and
an inner sheath having a proximal end and a distal end and being sized relative to said hollow outer sheath to permit said inner sheath to be selectively rotated and axially moved within said hollow outer sheath such that said distal end of said inner sheath may selectively protrude beyond said distal end of said hollow outer sheath and wherein said inner sheath has at least one working channel formed therein.

2. A guide system according to claim 1 further comprising at least one seal between said hollow outer sheath and said inner sheath.

3. A guide system according to claim 2 further comprising at least one other seal associated with each said working channel in said inner sheath.

4. A guide system according to claim 1 wherein at least one of said working channels has a substantially oblong cross-sectional shape.

5. A guide system according to claim 1 wherein said inner sheath operably supports a camera therein.

6. A guide system according to claim 1 wherein said inner sheath operably supports at least one endoscopic tool selected from the group of endoscopic tools consisting of: surgical lights, suction hoses, and fluid-supply hoses, imaging devices, cameras, graspers, clip appliers, loops, radio frequency ablation devices, harmonic ablation devices, scissors, knives, and suturing devices.

7. A guide system according to claim 1 wherein said inner sheath is configured relative to said outer sheath to permit said inner sheath to be lockingly retained in at least one position relative to said outer sheath.

8. A guide system according to claim 7 further comprising:
at least one detent formed on one of said inner sheath and said hollow outer sheath; and
at least one detent receiving pocket formed in the other of said inner sheath and said outer sheath for receiving said at least one detent therein to retain said inner sheath in a desired position relative to said hollow outer sheath.

9. A method for processing an instrument for surgery, the method comprising:
obtaining the guide system of claim 1;
sterilizing the guide system; and
storing the guide system in a sterile container.

10. A guide system for operably supporting endoscopic tools, comprising:
a hollow outer sheath having a proximal end and a distal end, said distal end being substantially steerable; and
a plurality of inner sheaths wherein each said inner sheath may be selectively inserted into said outer sheath one at a time and wherein each said inner sheath has a proximal end and a distal end and is sized relative to said outer sheath to permit said inner sheath to be selectively rotated and axially moved within said hollow outer sheath such that said distal end of said inner sheath may selectively protrude beyond said distal end of said outer sheath and wherein said inner sheaths are differently configured from each other to operably support different tool configurations therein.

11. The guide system according to claim 10 further comprising means for establishing a seal between any one of said inner sheaths and said hollow outer sheath when said anyone of said inner sheaths is inserted into said hollow outer sheath.

12. A method for performing a surgical procedure on a patient, comprising:
inserting the hollow outer sheath of claim 1 through a natural body lumen;
inserting an endoscope through the hollow outer sheath to identify a particular target tissue within the body;
inserting a hole-forming instrument through a working channel in said endoscope to form a hole through the target tissue;
removing the endoscope from the outer sheath;
inserting an inner sheath into the hollow outer sheath;
manipulating the inner sheath within the hollow outer sheath to orient a distal end of the inner sheath in a desired orientation;
insufflating a portion of a body cavity adjacent the target tissue; and
inserting at least one endoscopic tool through a corresponding working channel in the inner sheath such that the endoscopic tool may be moved relative to the inner sheath while maintaining the insufflation within the body cavity portion.

13. The method of claim 12 wherein said insufflating comprises introducing an insufflation medium through said inner sheath into the body while maintaining a substantially airtight seal between the inner sheath and outer sheath to insufflate the body cavity portion adjacent the target tissue.

14. The method of claim 13 further comprising locking the inner sheath in a desired position relative to the outer sheath.

15. The method of claim 12 wherein said inserting an inner sheath comprises inserting the inner sheath having a camera therein.

16. The method of claim 15 further comprising moving the distal end of the hollow outer sheath and/or the distal end of the inner sheath as necessary.

17. The method of claim 16 further comprising rotating the camera relative to the inner sheath; and
axially moving the camera within the inner sheath such that a distal end of the camera protrudes out of the distal end of the inner sheath.

18. The method of claim 16 wherein said moving the distal end of the inner sheath comprises rotating the inner sheath relative to the hollow outer sheath.

19. The method of claim 16 wherein said moving the distal end of the inner sheath comprises axially moving the inner sheath within the hollow outer sheath such that the distal end of the inner sheath protrudes beyond the distal end of the hollow outer sheath.

20. The method of claim 16 wherein said moving comprises moving the inner sheath relative to the hollow outer sheath such that a picture created by the camera is oriented in a desired orientation on a screen communicating with the camera.

21. A surgical kit comprising:
an endoscope;
a hollow outer sheath having a proximal end and a distal end, said distal end being substantially steerable; and
at least one an inner sheath having a proximal end and a distal end and being sized relative to said hollow outer sheath to permit said inner sheath to be selectively rotated and axially moved within said hollow outer sheath such that said distal end of said inner sheath may selectively protrude beyond said distal end of said hollow outer sheath and wherein said inner sheath has at least one working channel formed therein.
